(19) United States
(12) Patent Application Publication
(10) Pub. No.: US 2014/0370525 A1
(43) Pub. Date: Dec. 18, 2014

(54) ALPHA-HEMOGLOBIN STABILIZING PROTEIN ANTIBODIES AND METHODS OF USE THEREOF

(71) Applicant: The Children'a Hospital of Philadelphia, Philadelphia, PA (US)

(72) Inventors: Mitchell Weiss, Wynnewood, PA (US); John Kim Choi, Germantown, TN (US)

(73) Assignee: The Children's Hospital of Philadelphia, Philadelphia, PA (US)

(21) Appl. No.: 14/352,343

(22) PCT Filed: Oct. 18, 2012
(86) PCT No.: PCT/US12/60751

§ 371 Date: Apr. 17, 2014

Related U.S. Application Data
(60) Provisional application No. 61/548,437, filed on Oct. 18, 2011.

(51) Int. Cl. G01N 33/569

(52) U.S. Cl. CPC G01N 33/56966; G01N 2333/70582; G01N 2800/22
USPC 435/7.25

(57) ABSTRACT

Methods for detecting and/or imaging erythrocyte precursor cells are provided.

This application claims priority under 35 U.S.C. §119(e) to U.S. Provisional Patent Application No. 61/548,437, filed Oct. 18, 2011. The foregoing application is incorporated by reference herein.

This invention was made with government support under grant Nos. R01 DK61692 and R01 HL087427 awarded by the National Institutes of Health. The government has certain rights in the invention.

### FIELD OF THE INVENTION

The present invention relates to the field of immunology and hematology. Specifically, alpha-hemoglobin-stabilizing-protein (AHSP) antibodies and methods of use thereof are disclosed.

### BACKGROUND OF THE INVENTION

Several publications and patent documents are cited throughout the specification in order to describe the state of the art to which this invention pertains. Each of these citations is incorporated herein by reference as though set forth in full.

Currently, nucleated erythroid precursor cells are identified by morphology on hematoxylin and eosin stain and verified by immunohistochemical stains using antibodies against glycophorin A, glycophorin C, and hemoglobin. The current antibodies are unable to differentiate between nucleated erythroid precursors and mature non-nucleated red blood cells. Antibodies against transferrin receptor (CD71) are also used to detect nucleated erythroid precursors, but these antibodies also react nonspecifically with a variety of other cells, including tumor cells. An antibody which could better distinguish between precursor and mature red blood cells and nonerythroid cells would be useful for at least research and clinical testing of various blood disorders such as anemia, leukemia, and myelodysplasia.

### SUMMARY OF THE INVENTION

In accordance with the present invention, methods of determining the presence of a blood disorder, detecting an increased risk for a blood disorder, and/or diagnosing a blood disorder in a subject are provided. In a particular embodiment, the method comprises contacting a biological sample obtained from the subject with at least one antibody or antibody fragment immunologically specific for alpha-hemoglobin stabilizing protein (AHSP), and detecting cells bound by the AHSP antibody. The alteration in at least one morphological feature of the biological sample from the subject compared to a biological sample obtained from a normal subject is indicative of the blood disorder. The method may further comprise contacting the biological sample with other diagnostic agents or antibodies (e.g., at least one antibody to CD235, CD71, MPO, and/or hemoglobin). In a particular embodiment, the AHSP antibody is conjugated to at least one detection agent. In another embodiment, the AHSP antibody is detected by a secondary antibody (optionally conjugated to at least one detection agent). The blood disorder may be an anemia, leukemia, or myelodysplasias, such as a myelodysplastic syndrome (e.g., refractory cytopenia with multilineage dysplasia (RCMD)). The antibody may also be used to distinguish primary bone marrow disorders, as exemplified above, from other cancers that are metastatic to bone marrow. The antibody may also be used to detect placental nucleated erythroid precursors that are increased with perinatal hypoxia.

In accordance with another aspect of the instant invention, methods of detecting, imaging, and/or isolating erythroid precursor cells are provided. In a particular embodiment, the method comprises contacting a biological sample obtained from the subject with at least one antibody or antibody fragment immunologically specific for alpha-hemoglobin stabilizing protein (AHSP), and detecting, imaging, and/or isolating cells bound by the AHSP antibody. The method may further comprise contacting the biological sample with other diagnostic agents or antibodies (e.g., at least one antibody to CD235, CD71, MPO, and/or hemoglobin). In a particular embodiment, the AHSP antibody is conjugated to at least one detection agent. In another embodiment, the AHSP antibody is detected by a secondary antibody (optionally conjugated to at least one detection agent).

### BRIEF DESCRIPTION OF THE DRAWINGS

FIG. 1 provides images of AHSP stained normal (FIG. 1A) and myelodysplastic syndrome (FIG. 1B) bone marrow samples.

FIG. 2 provides images showing that AHSP and CD71 stain nucleated erythroid precursors (EPs). FIG. 2A provides an image of normal bone marrow by hematoxylin and eosin stain. FIG. 2B shows that CD235a stains both nucleated EPs and mature, anucleate red blood cells (RBCs). FIG. 2C shows AHSP stains nucleated EPs, but not mature, anucleate RBCs. FIG. 2D shows CD71 stains nucleated EPs, but not mature, anucleate RBCs. FIG. 2E provides an image of spleen with extramedullary hematopoiesis (hematoxylin and eosin). FIG. 2F shows AHSP stains nucleated EPs in splenic extramedullary hematopoiesis. The insets are high-magnification views of a representative section of the larger images.

FIG. 3 provides images which show that AHSP and CD71 stain erythroid blasts in acute erythroleukemia. FIG. 3A shows acute erythroleukemia by hematoxylin and eosin stain. FIG. 3B shows CD235a stains erythroid blasts and mature, anucleate RBCs. FIG. 3C shows AHSP stains erythroid blasts. FIG. 3D shows CD71 stains erythroid blasts.

FIG. 4 provides images showing CD71 stains myeloid blasts in acute myeloid leukemia, whereas AHSP does not. AHSP stains residual EPs and not myeloid blasts in acute myeloid leukemia with minimal differentiation (FIG. 4A), whereas CD71 stains both myeloid blasts and EPs (FIG. 4C). AHSP does not stain myeloid blasts in acute myelomonocytic leukemia (FIG. 4B), whereas CD71 does (FIG. 4D). FIGS. 4E and 4F provide corresponding hematoxylin and eosin-stained images.

FIG. 5 provides images which show that AHSP and CD71 stain megakaryocytes in primary myelofibrosis. FIG. 5A shows primary myelofibrosis by hematoxylin and eosin staining. FIG. 5B shows CD235a stains both nucleated EPs and mature, anucleate RBCs. AHSP (FIG. 5C) and CD71 (FIG. 5D) variably stain megakaryocytes and also stain nucleated EPs.

FIG. 6A shows AHSP stains residual EPs and not lymphoma cells in diffuse large B-cell lymphomas (DLBCLs). However, FIG. 6B shows CD71 stains both lymphoma cells and EPs. FIG. 6C provides a corresponding hematoxylin and eosin-stained slide. AHSP (FIG. 6D) does not stain metastatic carcinoma, whereas CD71 (FIG. 6E) does. FIG. 6F provides a corresponding hematoxylin and eosin-stained slide.

FIG. 7 provides images that giant pronormoblasts are evident in parvoviral infection. FIG. 7A provides a hematoxylin and eosin stain. FIG. 7B shows CD235a does not stain giant pronormoblasts. AHSP (FIG. 7C) and CD71 (FIG. 7D) stain giant pronormoblasts.

FIGS. 8A and 8B provide the immunohistochemical expression of AHSP, CD71, and CD235a in bone marrow and splenic specimens. The number in each column refers to the number of specimens with positive staining for each category of cells. *: Giant pronormoblasts are considered lesional cells for the purposes of this table; AML: acute myeloid leukemia; NA: not applicable; and NOS: not otherwise specified.

### DETAILED DESCRIPTION OF THE INVENTION

Alpha-hemoglobin-stabilizing-protein (AHSP) is a 12 kD chaperone protein which binds to the free alpha globin chain of hemoglobin, thereby preventing its aggregation and facilitating its incorporation into hemoglobin A. AHSP is a very abundant protein expressed specifically in red blood cell precursors and downregulated as these precursors mature. Identifying erythroid precursors is important in the analysis of bone marrow biopsies, particularly those that are diagnostically challenging such as in myelodysplastic syndromes (MDS). Although several erythroid markers are commercially available, they either stain both mature and immature erythroid precursors (glycophorin A, hemoglobin) or are not specific markers of the erythroid lineage (CD71). With these currently used antibodies, staining of mature erythrocytes, which come from the circulation, can obscure analysis and assessment of red blood cell production in the resident tissues, most commonly bone marrow and spleen. Herein, antibodies developed against AHSP were determined to specifically identify nucleated red blood cell precursors, but not mature erythrocytes, with high sensitivity and specificity, even in bone marrow biopsies of normal controls and patients with MDS.

Alpha-hemoglobin stabilizing protein (AHSP) is also known as erythroid differentiation related factor (EDRF) or erythroid associated factor (ERAF). AHSP is a highly conserved protein in humans, pigs, cows, rats, and mice. Feng et al. (Cell (2004) 119:629-640) provide amino acid sequences of human, cow, pig, rat, and mouse AHSPs (see also U.S. Patent Application Publication No. 20050028229). Gene ID: 51327 provides an example of human AHSP. Examples of other AHSP sequences include, without limitation, GenBank Accession Nos. include NP_{—}057717.1 (human AHSP amino acid sequence), NM_{—}016633.2 and AF485325 (human AHSP nucleotide sequence), AF485327 (Mus musculus), AF485326 (Bos tarus), and NM_{—}001106299 (Rattus Norvegicus). An exemplary amino acid sequence of human AHSP is:

(SEQ ID NO: 1)
MALLKANKDL ISAGLKEFSV LLNQQVFNDP LVSEEDMVTV
VEDWMNFYIN YYRQQVTGEP QERDKALQEL RQELNTLANP
FLAKYRDFLK SHELPSHPPP SS.

In accordance with the instant invention, AHSP antibodies and methods of use thereof are provided. Specifically, methods for the immunodetection and/or imaging of erythroid precursors and methods of targeting the same are provided. The antibodies can be used to monitor endogenous red cell production (erythropoiesis). The antibodies can also be used to detect blood disorders of erythroid development such as anemias, leukemias (e.g., erythroleukemia, pure erythroid leukemia, acute erythroid leukemia) and myeloproliferative disorders such as myelodysplasias (e.g., myelodysplastic syndromes (MDS)) and polycythemia vera. The antibodies may be used to distinguish these diseases from non-blood cancers that may cause anemia by invading the bone marrow. In a particular embodiment, the antibodies may be used to diagnose non-erythroid disorders such as, without limitation, myeloid/lymphoid leukemias, lymphomas, and metastatic cancer by excluding blood disorders of erythroid development. AHSP will also stain erythroid precursors in various non-malignant diseases such as thalassemia, sickle cell anemia and other hemolytic anemias, although these diseases rarely present diagnostic dilemmas and are not usually diagnosed by immunohistochemistry of bone marrow. The antibodies may be used as part of a panel of antibodies for the general analysis/diagnosis of biological samples, e.g., bone marrow biopsies. For example, the AHSP antibodies may be used to supplement or replace CD71/glycophorin antibodies.

In a particular embodiment, the antibodies of the instant invention can be used for detecting nucleated erythroid precursors in the placenta and/or umbilical cord blood, wherein the presence of nucleated erythroid precursors correlates with perinatal asphyxia and/or fetal hypoxia/stress. Indeed, detection of nucleated erythroid precursors using antibodies against AHSP in placental sections can be used as a surrogate marker of perinatal hypoxia. Elevated levels of umbilical cord nucleated red blood cells (nRBCs) or placental nRBCs have been used to assess in utero hypoxia (Bryant et al. (2006) J. Maternal-Fetal Neonatal Med., 19:105-108). The presence of perinatal hypoxia may be indicative of adverse neurologic outcomes (e.g., brain injury, cerebral palsy, etc.) (Redline, RW (2008) Pediatric Develop. Path. 11:456-464).

The antibodies of the instant invention may be antibodies or antibody fragments which are immunologically specific for AHSP, particularly human AHSP. The antibody may be monoclonal or polyclonal. A polyclonal antibody is described hereinbelow. An example of a monoclonal AHSP antibody is described in Morrison et al. (Hybridoma (2011) 30:175-179). Antibody fragments include, without limitation, immunoglobulin fragments such as single domain (Dab; e.g., single variable light or heavy chain domain), Fab, Fab′, F(ab′)₂, and F(v); and fusions (e.g., via a linker) of these immunoglobulin fragments including, without limitation: scFv, scFv₂, scFv-Fc, minibody, diabody, triabody, and tetrabody. Methods for recombinantly producing antibodies are well-known in the art. Indeed, commercial vectors for certain antibody and antibody fragment constructs are available. The instant invention also encompasses fusion proteins comprising at least one antibody or antibody fragment. The instant invention also encompasses synthetic proteins which mimic an immunoglobulin. Examples include, without limitation, Affibody® molecules (Affibody, Bromma, Sweden), darpins (designed ankyrin repeat proteins; Kawe et al. (2006) J. Biol. Chem., 281:40252-40263), and peptabodies (Terskikh et al. (1997) PNAS 94:1663-1668). Further, while the instant application describes antibodies to AHSP, any compound or protein which specifically recognizes or binds AHSP (particularly to the exclusion of other proteins) is encompassed by the instant invention.

Compositions comprising the antibodies (or fragments thereof) are also encompassed by the instant invention. In a particular embodiment, the composition comprises at least one antibody of the instant invention and at least one pharmaceutically acceptable carrier.

The antibodies of the instant invention may be further modified. For example, the antibodies may be humanized. The antibodies of the instant invention may also be conjugated/linked to other components. For example, the antibodies may be operably linked (e.g., covalently linked, optionally, through a linker) to at least one detectable agent, imaging agent, contrast agent, therapeutic agent, cytotoxic molecule, and/or any other compound. The antibodies of the instant invention may also comprise at least one purification tag (e.g., a His-tag).

The antibodies of the instant invention may also be linked to other antibodies (e.g., to generate scFv-scFv). For example, the antibodies of the instant invention may be linked to another antibody to generate bispecific antibodies. The antibodies may be the same or may be immunologically specific for the same protein (optionally different epitopes) or different proteins. The antibodies may be linked together as a fusion protein, optionally via a linker domain (e.g., from about 1 to about 100 amino acids). The antibodies may be linked together via a carrier molecule (e.g., human serum albumin). The antibodies may also be linked together via “knobs into holes” engineering (e.g., preferentially pairing of light and heavy chains; see, e.g., Ridgway et al. (1996) Protein Eng. (1996) 9:617-621).

The antibody molecules of the invention may be prepared using a variety of methods known in the art. Antibodies may be prepared by chemical cross-linking, hybrid hybridoma techniques and by expression of recombinant antibody or antibody fragments expressed in host cells, such as mammalian cells, bacteria or yeast cells. In one embodiment of the invention, the antibody molecules are produced by expression of recombinant antibody or antibody fragments in host cells. The nucleic acid molecules encoding the antibody may be inserted into expression vectors and introduced into host cells. The resulting antibody molecules are then isolated and purified from the expression system. The antibodies optionally comprise a purification tag by which the antibody can be purified.

The purity of the antibody molecules of the invention may be assessed using standard methods known to those of skill in the art, including, but not limited to, ELISA, immunohistochemistry, ion-exchange chromatography, affinity chromatography, immobilized metal affinity chromatography (IMAC), size exclusion chromatography, polyacrylamide gel electrophoresis (PAGE), western blotting, surface plasmon resonance and mass spectroscopy.

AHSP antibodies (or fragments thereof) have broad applications in therapy and diagnosis. Specifically, the AHSP antibody molecules of the invention may be used, for example: (1) to isolate, detect, and/or image erythroid precursor cells; (2) as a diagnostic tool; and (3) to deliver compounds to erythroid precursor cells (e.g., any natural or synthetic chemical compounds (such as small molecule compounds (a substance or compound that has a relatively low molecular weight (e.g., less than 4,000 atomic mass units (a.m.u.), particularly less than 2,000 a.m.u.), organic or inorganic compounds and molecules, biological macromolecules (such as saccharides, lipids, peptides, proteins, polypeptides and nucleic acid molecules (e.g., those encoding a protein of interest), inhibitory nucleic acid molecule (e.g., antisense or siRNA), and drugs (e.g., an FDA approved drug)).

Erythroid precursor cells are those cells that give rise to erythrocytes by the process of erythropoiesis. Erythroid precursor cells are committed to the formation of erythrocytes and often have morphological features distinctive of the erythroid lineage. Erythroid precursor cells include pronormoblast (earliest morphologically recognized erythroid precursor cell), basophilic normoblast (early erythroid precursor cell), polychromatophilic normoblast (intermediate erythroid precursor cell), and orthochromatophilic normoblast (late erythroid precursor cell).

The AHSP antibody molecules of the instant invention can be administered to a patient, as described hereinbelow. The AHSP antibody molecules may be administered to a subject to deliver a therapeutic agent. The AHSP antibody molecules of the instant invention may be conjugated to, without limitation, cytotoxic molecules, therapeutic agents (e.g., chemotherapeutic agents), radioisotopes, pro-drugs, and pro-drug activating enzymes capable of converting a pro-drug to its active form. If the compound to be conjugated is proteinaceous, a fusion protein may be generated with the antibody molecule. Radiosensitizers may also be administered with the antibodies. In a particular embodiment, the AHSP antibodies are humanized. The AHSP antibodies may also be linked to a cell penetrating peptide to increase internalization such as the TAT leader sequence (e.g., Vives et al. (1997) J. Biol. Chem., 272:16010-7; Wadia et al. (2004) Nat. Med., 10:310-5).

When employed for detecting and/or imaging cells (e.g., erythroid precursor cells), the AHSP antibody molecules of the invention can be conjugated to detectable agents such as radioisotopes, imaging agent, and/or contrast agent as described hereinabove. The AHSP antibody molecules can be conjugated to the radioisotopes by any method including direct conjugation and by linking through a chelator. The AHSP antibody molecules may also be conjugated to labels or contrast agents such as, without limitation, paramagnetic or superparamagnetic ions for detection by MRI imaging, isotopes (e.g., radioisotopes (e.g., ³H (tritium) and ¹⁴C) or stable isotopes (e.g., ²H (deuterium), ¹¹C, ¹³C, ¹⁷O and ¹⁸O), optical agents, and fluorescence agents. Paramagnetic ions include, without limitation, Gd(III), Eu(III), Dy(III), Pr(III), Pa(IV), Mn(II), Cr(III), Co(III), Fe(III), Cu(II), Ni(II), Ti(III), and V(IV). Fluorescent agents include, without limitation, fluorescein and rhodamine and their derivatives. Optical agents include, without limitation, derivatives of phorphyrins, anthraquinones, anthrapyrazoles, perylenequinones, xanthenes, cyanines, acridines, phenoxazines and phenothiazines. In a particular embodiment, the AHSP antibodies are humanized. The AHSP antibodies may also be linked to a cell penetrating peptide.

The AHSP antibody molecules of the invention may also be used in gene therapy for direct targeting of vehicles (liposomes, viruses etc.) containing genes to erythroid precursor cells. In an exemplary embodiment, liposomes may be studded by the AHSP antibody molecules of the invention to facilitate erythrocyte precursor cell specific targeting. In another embodiment, AHSP antibodies may be expressed directly on the surface of viruses or as fusions with viral coat proteins to facilitate erythrocyte precursor cell specific targeting.

The AHSP antibody molecules of the invention may be used, for example, to 1) diagnose (e.g., identify and/or determine an increased risk of) a blood disorder in a patient, 2) determine the prognosis of a patient, including stage or status of a blood disorder and/or its potential sensitivity to therapy, and 3) determine the efficacy of a blood disorder treatment of a patient. In a particular embodiment, the AHSP antibody molecules are administered to a subject for the above purposes. In another particular embodiment, the AHSP antibody molecules are utilized to detect and/or image erythroid precursors cells in a biological sample from a patient. The biological sample may be, without limitation, a spleen biopsy, bone marrow, isolated blood cells, or blood. Many immunological assays are well known in the art for assaying of biological samples for the presence of a certain protein including, without limitation: immunohistochemistry, immunoprecipitations, radioimmunoassays, enzyme-linked immunosorbent assays (ELISA), immunohistochemical assays, Western blot and the like. Similar assays (e.g., immunoprecipitations) may be used to isolate/purify the erythroid precursor cells bound by AHSP antibody.

In a particular embodiment, the methods of the instant invention comprise contacting a biological sample obtained from a patient with at least one antibody (or fragment thereof) immunologically specific for AHSP and detecting the presence of the antibody (particularly after washing away unbound antibody). The sample may be contacted with at least one other diagnostic agent or antibody. The AHSP antibody may be detected directly (e.g., via an attached detection or imaging agent as described above) or indirectly (e.g., via a secondary antibody which recognizes the AHSP antibody (e.g., an anti-rabbit or anti-mouse antibody which is conjugated to a detection or imaging tag as described hereinabove)). The AHSP stained cells are erythroid precursor cells. This identifies, for example, acute erythroid leukemias from other leukemias and metastatic tumors. The presence of at least one morphological feature in the AHSP-stained cells/tissue that is different than AHSP-stained cells/tissue from a normal, control subject is indicative of a blood disorder, such as a myelodysplastic syndrome. In a particular embodiment, the morphological feature is detected and analyzed by a computerized analysis algorithm. The morphological features include, without limitation, the relative abundance of nucleated erythroid precursors, the spatial arrangement of nucleated erythroid precursors, cellularity, or size, morphology, or cellular architecture of erythroid cluster.

The present invention further encompasses kits for use in detecting the expression of AHSP and identifying erythroid precursor cells, e.g., in biological samples. Such kits may comprise the AHSP antibody molecules of the invention (particularly in at least one carrier) as well as buffers and other compositions and instruction material. The AHSP antibody molecule may be conjugated to labels or contrast agents as described hereinabove. The kits may further comprise other agents and/or antibodies for the detection and/or identification of blood cells and/or blood disorders (e.g., diagnostic antibodies). When additional antibodies or other markers are included in the kit, the antibodies or markers may comprise a detection agent different than the one attached to the AHSP antibody (if present). For example, the kit may comprise ASHP and CD235 antibodies wherein the antibodies are linked to different fluorescent molecules of different colors permitting the detection of nucleated erythroid cells and mature non nucleated RBCs at the same time. In a particular embodiment, the kit comprises agents for hematoxylin and eosin staining (e.g., hemalum and eosin (e.g., eosin Y)). In a particular embodiment, the kit may also comprise at least one antibody to CD235, CD71, and/or hemoglobin. In a particular embodiment, the kit comprises AHSP antibodies and CD235 antibodies. The kits of the instant invention may also comprise an antibody to myeloperoxidase (MPO). The inclusion of the MPO antibodies allows for detection of erythroid precursor cells and maturing myeloid cells at the same time, thereby permitting a reliable detection of M:E ratio.

The antibodies as described herein will generally be administered to a patient as a pharmaceutical preparation. The term “patient” as used herein refers to human or animal subjects. These antibodies may be employed therapeutically, under the guidance of a physician for the treatment of blood disorders, or diagnostically. The pharmaceutical preparation comprising the antibody molecules of the invention may be conveniently formulated for administration with at least one pharmaceutically acceptable carrier, such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of antibody molecules in the chosen medium will depend on the hydrophobic or hydrophilic nature of the medium, as well as the size and other properties of the antibody molecules. Solubility limits may be easily determined by one skilled in the art.

As used herein, “pharmaceutically acceptable carrier” includes any and all solvents, dispersion media and the like which may be appropriate for the desired route of administration of the pharmaceutical preparation, as exemplified in the preceding paragraph. The use of such media for pharmaceutically active substances is known in the art. Except insofar as any conventional media or agent is incompatible with the antibody molecules to be administered, its use in the pharmaceutical preparation is contemplated.

The dose and dosage regimen of an antibody according to the invention that is suitable for administration to a particular patient may be determined by a physician considering the patient's age, sex, weight, general medical condition, and the specific condition and severity thereof for which the antibody is being administered. The physician may also consider the route of administration of the antibody, the pharmaceutical carrier with which the antibody may be combined, and the antibody's biological activity.

Selection of a suitable pharmaceutical preparation depends upon the method of administration chosen. For example, the antibodies of the invention may be administered intravenously or by direct injection. Pharmaceutical preparations for intravenous injection are known in the art. If a small form of the antibody is to be administered, including but not limited to a Fab fragment, a Dab, an scFv or a diabody, it may be conjugated to a second molecule such as, but not limited to polyethylene glycol (PEG) or an albumin-binding antibody or peptide to prolong its retention in blood.

### DEFINITIONS

The following definitions are provided to facilitate an understanding of the present invention:

Myelodysplastic syndromes (MDS) are a group of disorders characterized by one or more peripheral blood cytopenias secondary to bone marrow dysfunction. The syndromes may arise de novo, or following treatment with chemotherapy and/or radiation therapy. Typically, the bone marrow of subjects with a myelodysplastic syndrome shows qualitative and quantitative changes suggestive of a preleukemic process, but having a chronic course that does not necessarily terminate as acute leukemia.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. An isolated nucleic acid (either DNA or RNA) may further represent a molecule produced directly by biological or synthetic means and separated from other components present during its production.

A “vector” is a nucleic acid molecule such as a plasmid, cosmid, bacmid, phage, or virus, to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The term “substantially pure” refers to a preparation comprising at least 50-60% by weight of a given material (e.g., nucleic acid, oligonucleotide, polypeptide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

The phrase “operably linked,” as used herein, may refer to a nucleic acid or amino acid sequence placed into a functional relationship with another nucleic acid or amino acid sequence. Examples of nucleic acid sequences that may be operably linked include, without limitation, promoters, cleavage sites, purification tags, transcription terminators, enhancers or activators and heterologous genes which when transcribed and translated will produce a functional product such as a protein, ribozyme or RNA molecule.

An “antibody” or “antibody molecule” is any immunoglobulin, including antibodies and fragments thereof, that binds to a specific antigen. As used herein, antibody or antibody molecule contemplates intact immunoglobulin molecules, immunologically active portions of an immunoglobulin molecule, and fusions of immunologically active portions of an immunoglobulin molecule.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

“Fv” is an antibody fragment which contains an antigen-recognition and -binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although often at a lower affinity than the entire binding site.

“Single-chain Fv” or “scFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen binding.

As used herein, the term “immunotoxin” refers to chimeric molecules in which antibody molecules or fragments thereof are coupled or fused (i.e., expressed as a single polypeptide or fusion protein) to toxins or their subunits. Toxins to be conjugated or fused can be derived form various sources, such as plants, bacteria, animals, and humans or be synthetic toxins (drugs), and include, without limitation, saprin, ricin, abrin, ethidium bromide, diptheria toxin, Pseudomonas exotoxin, PE40, PE38, saporin, gelonin, RNAse, protein nucleic acids (PNAs), ribosome inactivating protein (RIP), type-1 or type-2, pokeweed anti-viral protein (PAP), bryodin, momordin, and bouganin.

The term “conjugated” refers to the joining by covalent or noncovalent means of two compounds or agents of the invention.

The term “pro-drug” refers to a compound which is transformed in vivo to an active form of the drug. The pro-drug may be transformed to an active form only upon reaching the target in vivo or upon internalization by the target cell.

Radioisotopes of the instant invention include, without limitation, positron-emitting isotopes and alpha-, beta-, gamma-, Auger- and low energy electron-emitters. The radioisotopes include, without limitation: ¹³N, ¹⁸F, ³²P, ⁶⁴Cu, ⁶⁶Ga, ⁶⁷Ga, ⁶⁸Ga, ⁶⁷Cu, ⁷⁷Br, ⁸⁰Br, ⁸²Rb, ⁸⁶Y, ⁸⁹Zr, ⁹⁰Y, ⁹⁵Ru, ⁹⁷Ru, ⁹⁹Tc, ¹⁰³Ru, ¹⁰⁵Ru, ¹²⁶In, ^{113m}In, ¹¹³Sn, ^{121m}Te, ^{122m}Te, ^{125m}Te, ¹²³I, ¹²⁴I, ¹²⁸I, ¹²⁶I, ¹³¹I, ¹³³I, ¹⁶⁵Tm, ¹⁶⁷Tm, ¹⁶⁸Tm, ¹⁷⁷Lu, ¹⁸⁶Re, ¹⁸⁸Re, ^{195m}Hg, ²¹¹At, ²¹²Bi, and ²²⁵Ac. When the conjugated antibodies of the instant invention are employed for radio-immunodetection, the radioisotope is preferably a gamma-emitting isotope. When the conjugated antibodies of the instant invention are employed for detection by ImmunoPET (positron emission tomography), the radioisotope is preferably a positron-emitting isotope such as, without limitation, ¹³N, ¹⁸F, ⁸⁹Zr, ⁸²Rb. When the conjugated antibodies of the instant invention are employed for radioimmunotherapy (i.e., the treating of a patient with cancer), the radioisotope is preferably selected from the group consisting of ⁹⁰Y, ¹³¹I, and ¹⁷⁷Lu, ¹⁸⁶Re, although other radionuclides such as many of those listed above are also suitable.

The term “radiosensitizer”, as used herein, is defined as a molecule administered to animals in therapeutically effective amounts to increase the sensitivity of the cells to radiation. Radiosensitizers are known to increase the sensitivity of cancerous cells to the toxic effects of radiation. Radiosensitizers include, without limitation, 2-nitroimidazole compounds, and benzotriazine dioxide compounds, halogenated pyrimidines, metronidazole, misonidazole, desmethylmisonidazole, pimonidazole, etanidazole, nimorazole, mitomycin C, RSU 1069, SR 4233, E09, RB 6145, nicotinamide, 5-bromodeoxyuridine (BUdR), 5-iododeoxyuridine (IUdR), bromodeoxycytidine, fluorodeoxyuridine (FudR), hydroxyurea, cisplatin, and therapeutically effective analogs and derivatives of the same.

As used herein, a linker is a chemical moiety comprising a covalent bond or a chain of atoms that covalently attaches two molecules to each other. In a particular embodiment, the linker may contain amino acids, particularly from 1 to about 25, 1 to about 20, 1 to about 15, 1 to about 10, or 1 to about 5 amino acids.

As used herein, a “biological sample” refers to a sample of biological material obtained from a subject, particularly a human subject, including a tissue, a tissue sample, a cell sample, a tumor sample, and a biological fluid (e.g., blood). In a particular embodiment, the biological sample is bone marrow.

As used herein, “diagnose” refers to detecting and identifying a disease/disorder in a subject. The term may also encompass assessing or evaluating the disease/disorder status (progression, regression, stabilization, response to treatment, etc.) in a patient known to have the disease/disorder.

As used herein, the term “prognosis” refers to providing information regarding the impact of the presence of a disease/disorder on a subject's future health (e.g., expected morbidity or mortality). In other words, the term “prognosis” refers to providing a prediction of the probable course and outcome of a disease/disorder or the likelihood of recovery from the disease/disorder.

The term “treat” as used herein refers to any type of treatment that imparts a benefit to a patient afflicted with a disease, including improvement in the condition of the patient (e.g., in one or more symptoms), delay in the progression of the condition, etc.

The terms “detection label” or “detection agent” refers to a detectable marker that may be detected by a physical or chemical means such as, without limitation, optical, electromagnetic, radiation, fluorescence, photonic, electronic, magnetic, or enzymatic means.

The term “diagnostic antibody” refers to an antibody that is used as a diagnostic reagent for a disease or disorder. The diagnostic antibody may bind to a target that is specifically associated with, or shows increased expression in, a particular disease or disorder. The diagnostic antibody may be used, for example, to detect a target in a biological sample from a patient, or in diagnostic imaging of disease sites in a patient.

The following examples provide illustrative methods of practicing the instant invention, and are not intended to limit the scope of the invention in any way.

### Example 1

Alpha-hemoglobin stabilizing protein (AHSP) is an abundant, erythroid-specific chaperone protein that binds nascent alpha-globin polypeptide to stabilize its native folding and facilitate its incorporation into hemoglobin A. Identification of erythroid precursors enables characterization of the topographic structure of progenitor cells in bone marrow biopsies. Architectural disruption of erythroid islands is thought to occur in myelodysplastic syndromes. However, this feature is subjective and difficult to formally quantitate, limiting its use for diagnostic studies. One current limitation in the field is that available antibodies against erythroid islands also stain contaminating mature red blood cells (anti-glycophorin) or some non erythroid tumor cells (anti-CD71). Compared to these reagents, AHSP antibody stains erythroid islands with greater specificity and sensitivity.

Antibodies against AHSP were generated by immunizing a rabbit with a full length recombinant human AHSP prepared in E. coli. Bone marrow biopsies from ten normal patients and ten patients with refractory cytopenia with multilineage dysplasia (RCMD) were identified by searching the laboratory information system. Biopsy specimens from all cases were stained for AHSP, CD71 (transferrin receptor protein 1), CD235 (glycophorin A), and hemoglobin. Blinded microscopic assessment of staining of non-nucleated erythroid cells and semi-quantitative assessment of percentage of erythroid cells was performed.

Microscopic analysis of immunohistochemical stains demonstrated that glycophorin A and hemoglobin stained both nucleated erythroid precursors and mature, non-nucleated red cells in normal and MDS bone marrow biopsies, consistent with known limitations of these markers. CD71 and AHSP both stained much less than 1% of all mature, non-nucleated red cells in normal and MDS bone marrow biopsies, although rare non-erythroid cells and non-nucleated red cells showed dim staining with CD71 but not with AHSP. Semi-quantitative measurement of the percentage of erythroid cells in bone marrow biopsies demonstrate a correlation between AHSP and CD71 in normal bone marrows (R²=0.66) and bone marrow biopsies from patients with MDS (R²=0.88). Thus, AHSP is a lineage-specific marker of nucleated erythroid precursors that performs favorably compared to commercially available antibodies.

AHSP expression was also characterized by immunohistochemistry in a panel of 85 neoplastic and reactive bone marrow biopsies. AHSP expression was compared to the staining patterns of the previously established erythroid markers CD71 and CD235a. AHSP immunohistochemistry was then used to study erythroid architectural disruption in bone marrow biopsies from cases of myelodysplastic syndromes and normal controls. The slides were digitized and a computerized image analysis algorithm was developed to identify AHSP-expressing cells, extract morphologic features of the biopsies (e.g., cellularity, size of erythroid clusters), and classify images as ‘MDS’ or ‘Normal’.

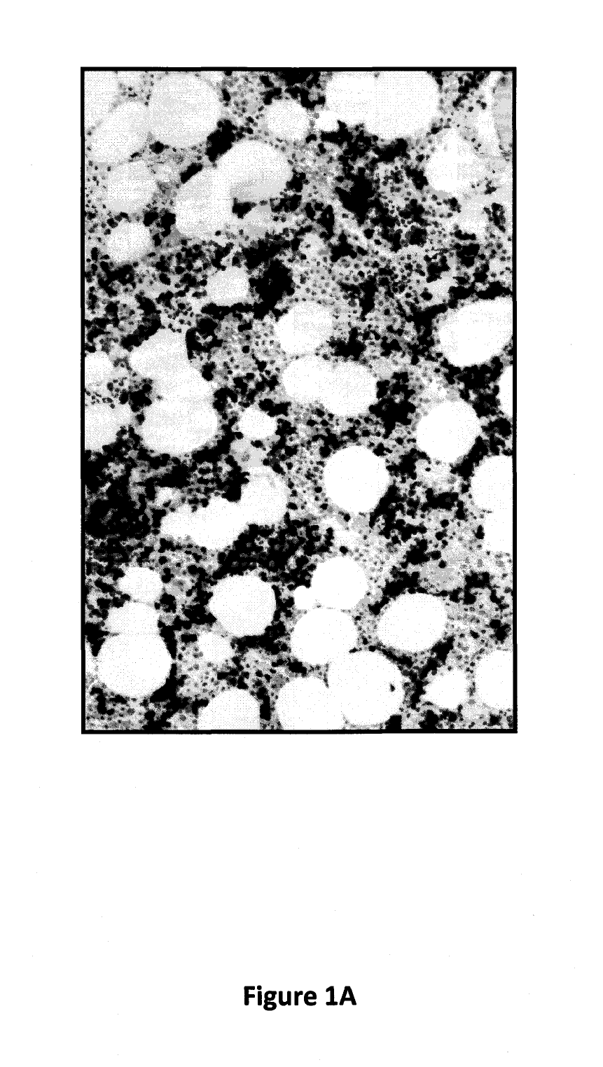

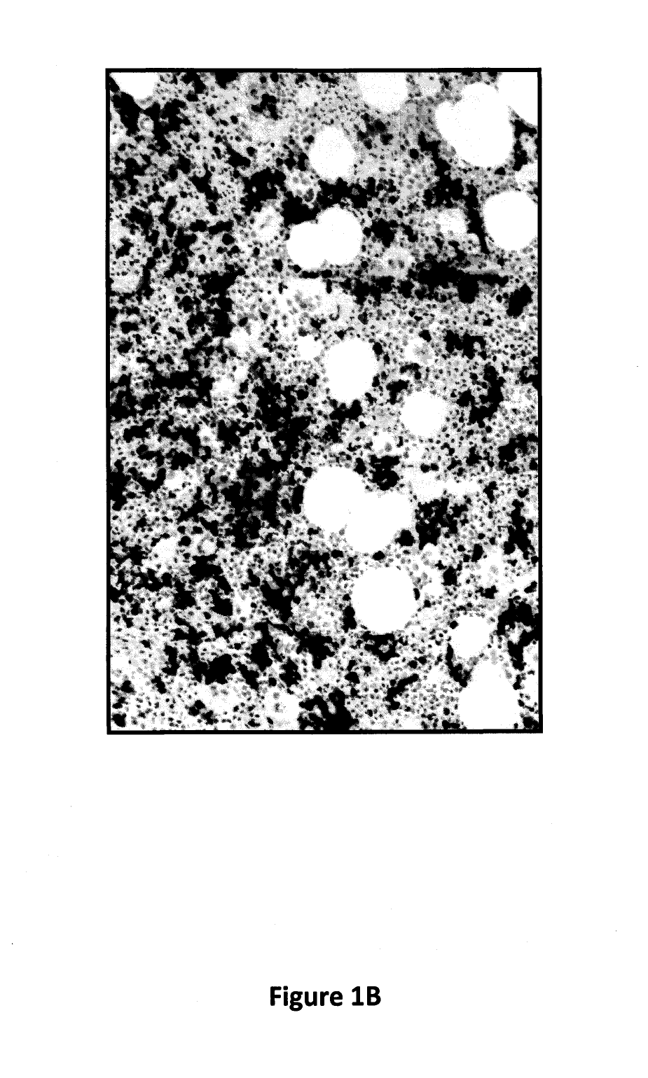

AHSP expression was limited to physiologic nucleated erythroid precursors in all control cases and blasts in erythroleukemia and pure erythroid leukemia. While CD71 also stained nucleated erythroid precursors in all of these samples, it additionally decorated non-erythroid blasts in many other cases of acute leukemia, diffuse large B cell lymphoma cells, and metastatic small cell carcinoma. Although CD71 staining of these cells was less intense than the staining seen in nucleated erythroid precursors, it was clearly above background and would interfere with the detection of nucleated erythroid precursors. CD235a stained both nucleated erythroid precursors and non-nucleated RBCs in all specimens, limiting its utility. FIG. 1 provides examples of AHSP stained cells from normal and MDS subjects.

Computerized image analysis of bone marrow biopsies from RCMD and normal cases identified cellularity and the size of AHSP-expressing erythroid clusters (as measured at the 85th percentile) to be the features that best discriminate cases of RCMD from normal. Receiver operating characteristic curves generated for these features demonstrate areas under the curve (AUCs) of 0.8875 for cellularity, 0.8661 for the size of erythroid clusters, and 0.9366 for the combination of the two features.

AHSP immunostaining recognizes nucleated erythroid precursors with increased specificity compared to CD71 and CD235, the most common erythroid markers currently used for clinical diagnostics. AHSP is superior to CD71 and CD235a for detecting normal and neoplastic nucleated erythroid precursors, including those found in erythroleukemia and MDS. Computerized image analysis of AHSP-stained bone marrow to assess erythroid architectural disruption and marrow cellularity can distinguish MDS from normal hematopoiesis.

### Example 2

Identification of erythroid precursors (EPs) in bone marrow biopsies is essential for lineage assignment of immature precursors or blasts, assessment of the myeloid:erythroid ratio, and evaluation of topographic features indicative of myelodysplasia. Morphologic assessment of hematoxylin and eosin-stained samples can help identify EPs in many circumstances. However, specimens with erythroid dyspoiesis, increased immature forms, topographic disarray, or nonhematopoietic lesions can be especially challenging and may require the use of immunohistochemical erythroid stains.

Antibodies currently used clinically to identify EPs include CD235a, CD71, and hemoglobin A (HbA). Each of these antibodies has specific limitations. CD235a and HbA label both EPs and mature, non-nucleated red blood cells (RBCs), creating excessive background staining in specimens with extensive hemorrhage. Furthermore, CD235a may not stain the most immature EPs (Sadahira et al. (2001) Int. J. Hematol., 74:147-152). CD71 [transferrin receptor 1 (TfR1)] has recently been shown to label EPs and not non-nucleated RBCs, potentially overcoming the limitations of CD235a (Dong et al. (2011) Am. J. Surg. Pathol., 35:723-732; Marsee et al. (2010) Am. J. Clin. Pathol., 134:429-435). However, CD71 expression occurs in a wide range of cells, such as activated T lymphocytes, T-lymphocyte and B-lymphocyte precursors, epithelial cells (including keratinocytes), and myocytes, and is generally considered to be a marker for rapidly proliferating cells (Ponka et al. (1999) Int. J. Biochem. Cell. Biol., 31:1111-1137). CD71 expression in bone marrow has been reported in acute myeloid and lymphoid leukemias and a variety of lymphomas involving the bone marrow. Thus, the nonspecific nature of CD71 could confound interpretation in diagnostically challenging cases or in limited samples. For these reasons, more sensitive and specific methods to identify EPs in clinical samples are needed to improve the accuracy of hematopathologic diagnoses. This need could be fulfilled by an antibody directed against an erythroid lineage-specific antigen that is highly expressed in EPs and downregulated upon their subsequent maturation into anucleate RBCs. Herein, it is shown that that α-hemoglobin-stabilizing protein (AHSP), an erythroid protein, fulfills these requirements.

AHSP is a 12 kDa chaperone protein that binds nascent α-globin and facilitates its incorporation into HbA (Kihm et al. (2002) Nature 417:758-763). AHSP binds reversibly with free α-globin, preventing its aggregation and stabilizing its structure before binding β-globin to form HbA (Kong et al. (2004) J. Clin. Invest., 114:1457-1466). Loss of AHSP expression in a murine model results in globin precipitation with ineffective erythropoiesis (Kong et al. (2004) J. Clin. Invest., 114:1457-1466; Favero et al. (2011) Biochem. Res. Int., 2011:373859). AHSP is expressed at high levels in lineage-committed EPs that are actively synthesizing Hb. In anucleate reticulocytes and mature RBCs, AHSP synthesis declines, and the protein is degraded. These properties make AHSP an ideal candidate for marking nucleated EPs.

Herein, AHSP expression was characterized in bone marrow biopsies and splenic specimens from adult and pediatric patients with a variety of hematopoietic neoplasms, metastatic nonhematopoietic cancers, and reactive conditions. Comparison with immunohistochemical staining of CD71 and CD235a on the same samples indicates that anti-AHSP staining is the superior approach for detecting EPs.

### Materials and Methods

All procedures were reviewed and approved by the Institutional Review Boards at the School of Medicine, University of Pennsylvania and the Children's Hospital of Philadelphia (CHOP).

Polyclonal antibody against full-length recombinant human AHSP was commercially prepared by Covance Research Products (Denver, Pa.).

Bone marrow biopsy and splenic specimens from reactive and neoplastic conditions were identified by searching the laboratory information systems of the Hospital of the University of Pennsylvania and CHOP. All adult biopsies were fixed in B5 and decalcified for 1 to 2 hours using RDO rapid decalcifying solution (Darlco, Oradell, N.J.). Pediatric bone marrow biopsies (for cases of neuroblastoma, rhabdomyosarcoma, primitive neuroectodermal tumor, and retinoblastoma) were fixed in acetic acid-zinc-formalin and decalcified for 30 minutes using RDO rapid decalcifying solution (Darlco, Oradell, N.J.). Splenic tissue was fixed in formalin. All samples were routinely embedded in paraffin.

Immunohistochemical staining was performed on 4-μm-thick sections for AHSP (rabbit polyclonal; 1:8000), CD71 (Invitrogen, Grand Island, N.Y.; H68.4; 1:1600), and CD235a (Dako, Carpinteria, Calif.; JC159; 1:1000). Deparaffinization, epitope retrieval with a pH9 buffer, and detection were performed on a Leica Bond-Max™ automated stainer. Staining was assessed visually in all specimens and categorized as “positive” or “negative” in each cell lineage, any neoplastic cells present, and non-cellular background material. All stainings were performed in the same laboratory with adequate positive and negative controls, and all samples were reviewed by 2 pathologists. Images included herein were obtained by scanning slides at x40 original magnification with an Aperio ScanScope™ CS. No color enhancement or alteration was performed on any image after scanning.

### Results

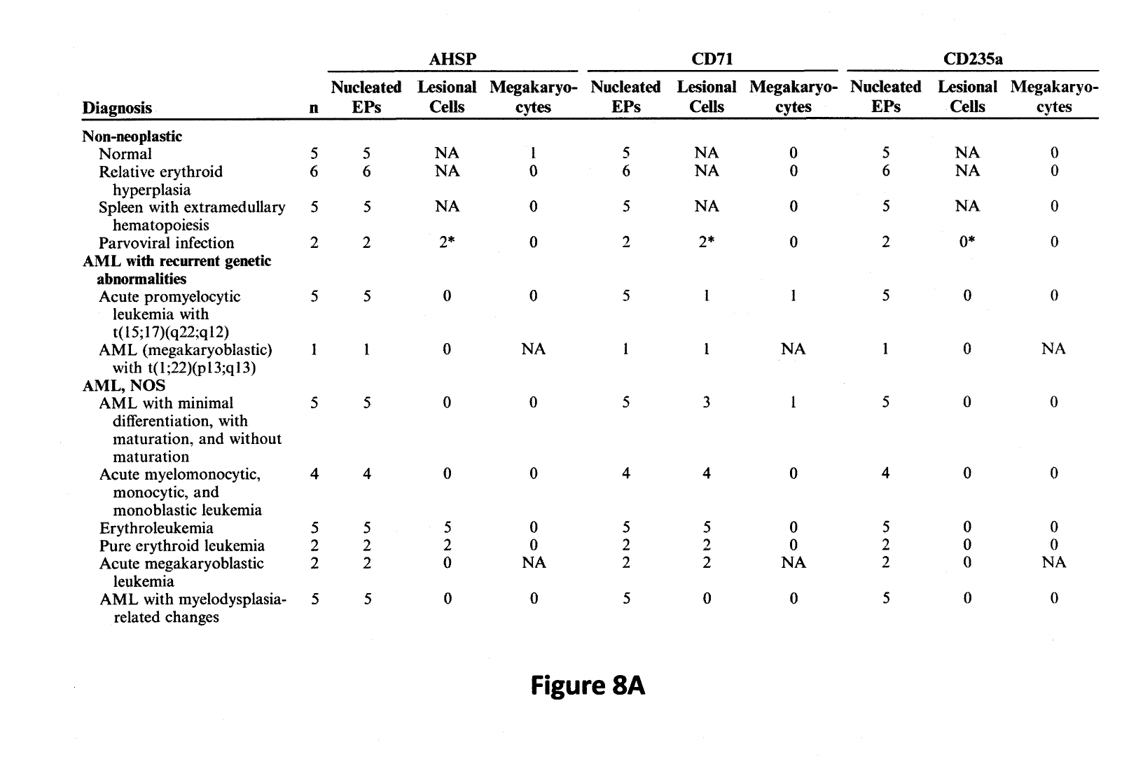

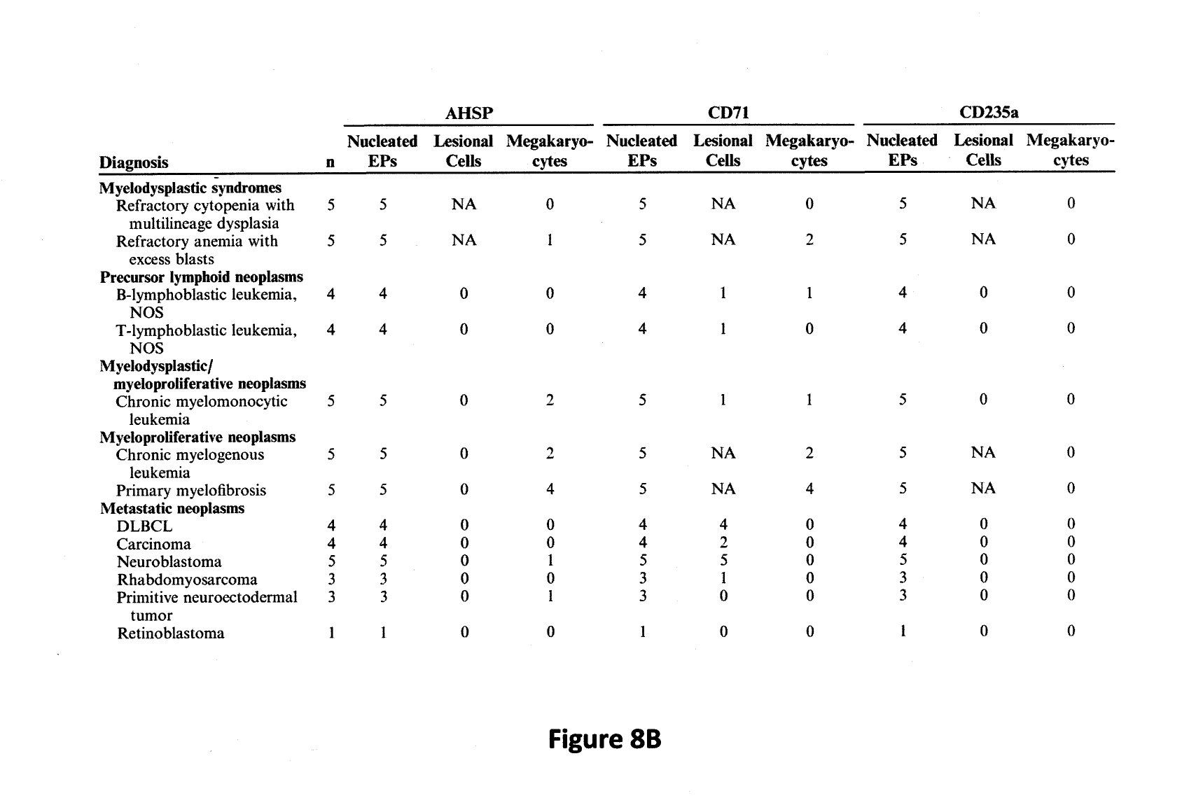

Immunohistochemical staining for AHSP, CD71, and CD235a was performed on 100 bone marrow samples representing a variety of neoplastic and reactive conditions and splenic tissue with extramedullary hematopoiesis; the results are detailed in FIG. 8. In summary, AHSP stained EPs in all specimens tested and did not stain nonerythroid cells. AHSP demonstrates a cytoplasmic staining pattern and brightly labels nucleated EPs from early (pronormoblast) through orthochromatic normoblast stages. CD235a stained both non-neoplastic EPs and mature (anucleate) RBCs, confounding evaluation in specimens with extensive hemorrhage and in splenic tissue with extramedullary hematopoiesis and congestion. Like AHSP, CD71 stained EPs in all specimens tested; however, it also stained neoplastic cells in a subset of nonerythroid acute leukemias, diffuse large B-cell lymphomas (DLBCLs), metastatic carcinomas, and small round blue cell tumors. Neither AHSP nor CD71 stained non-nucleated RBCs.

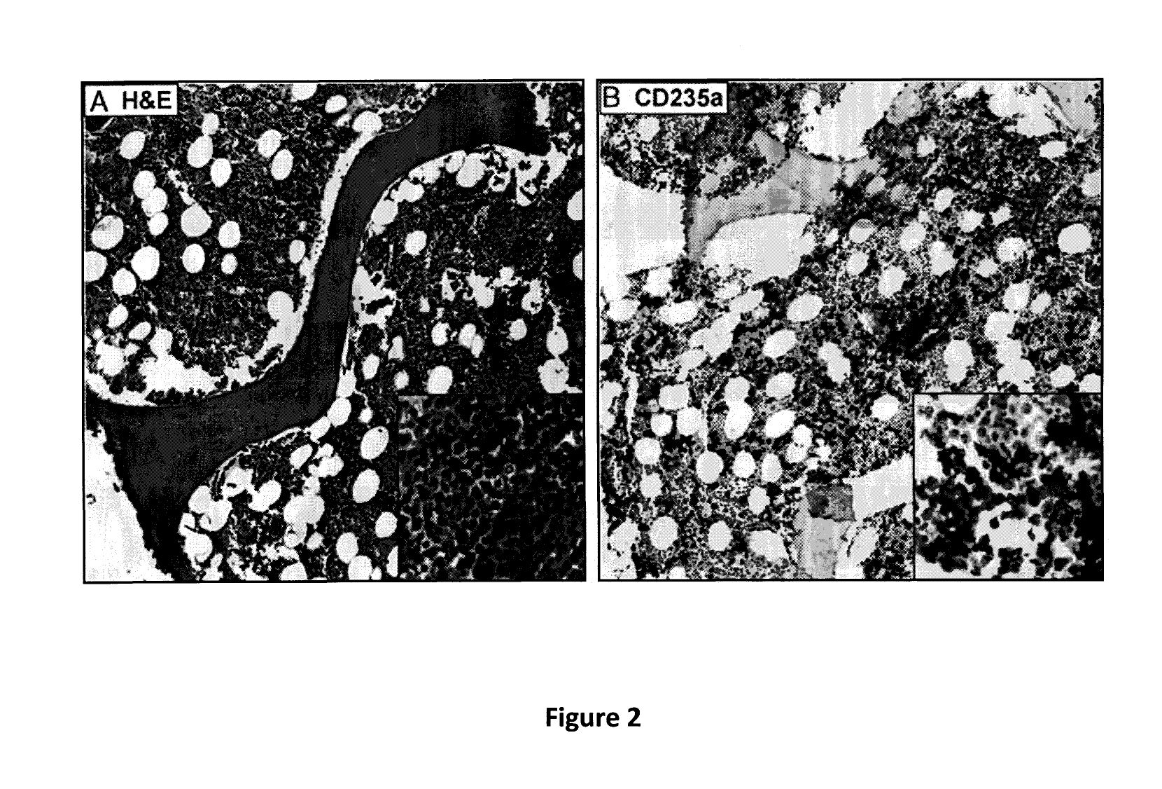

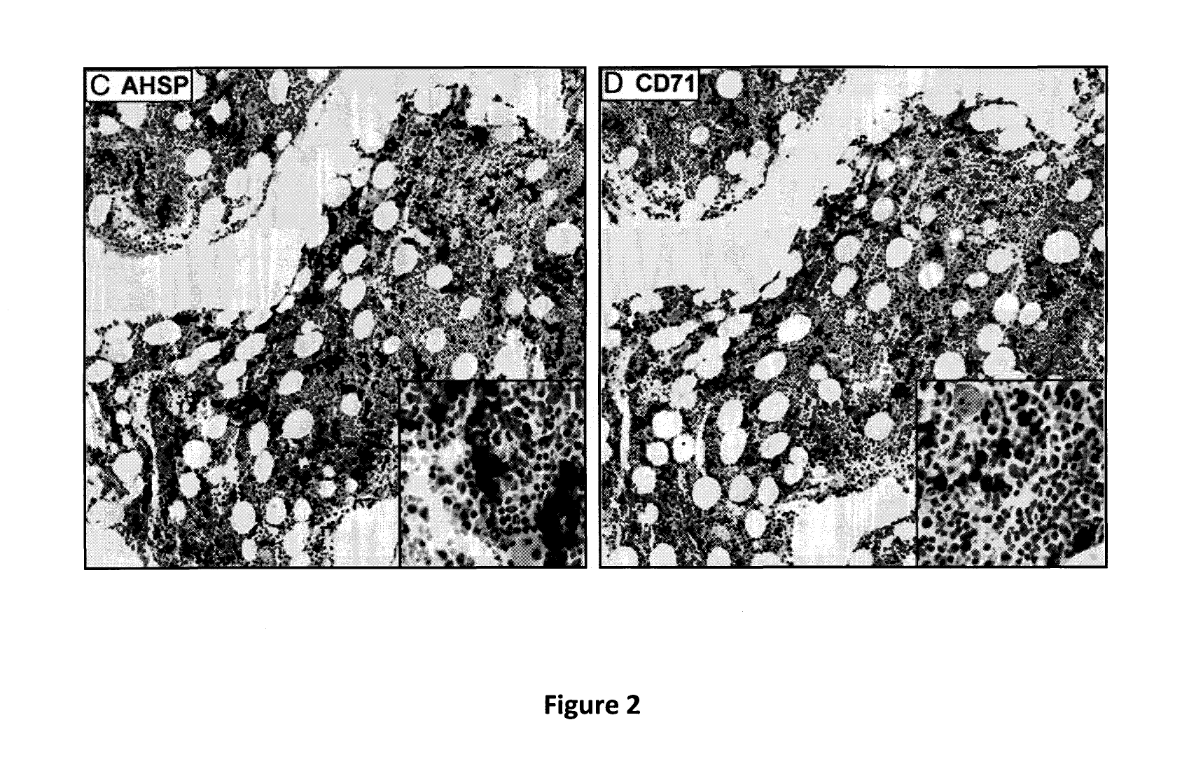

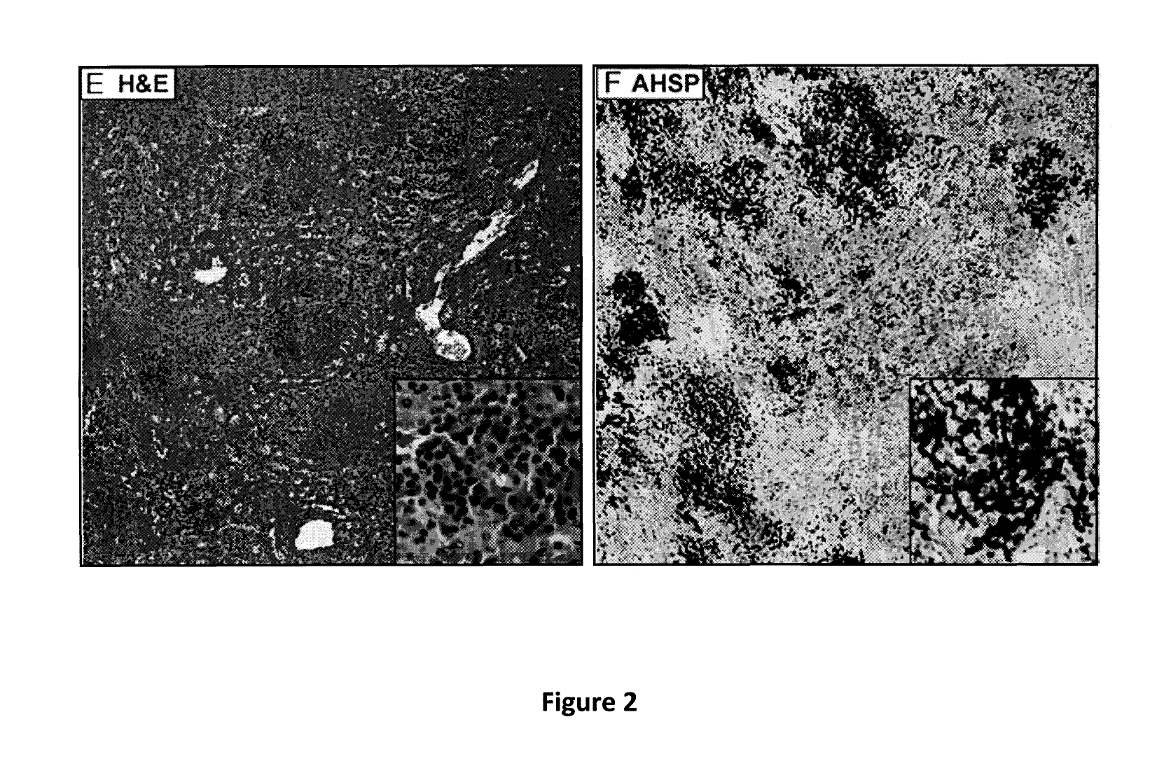

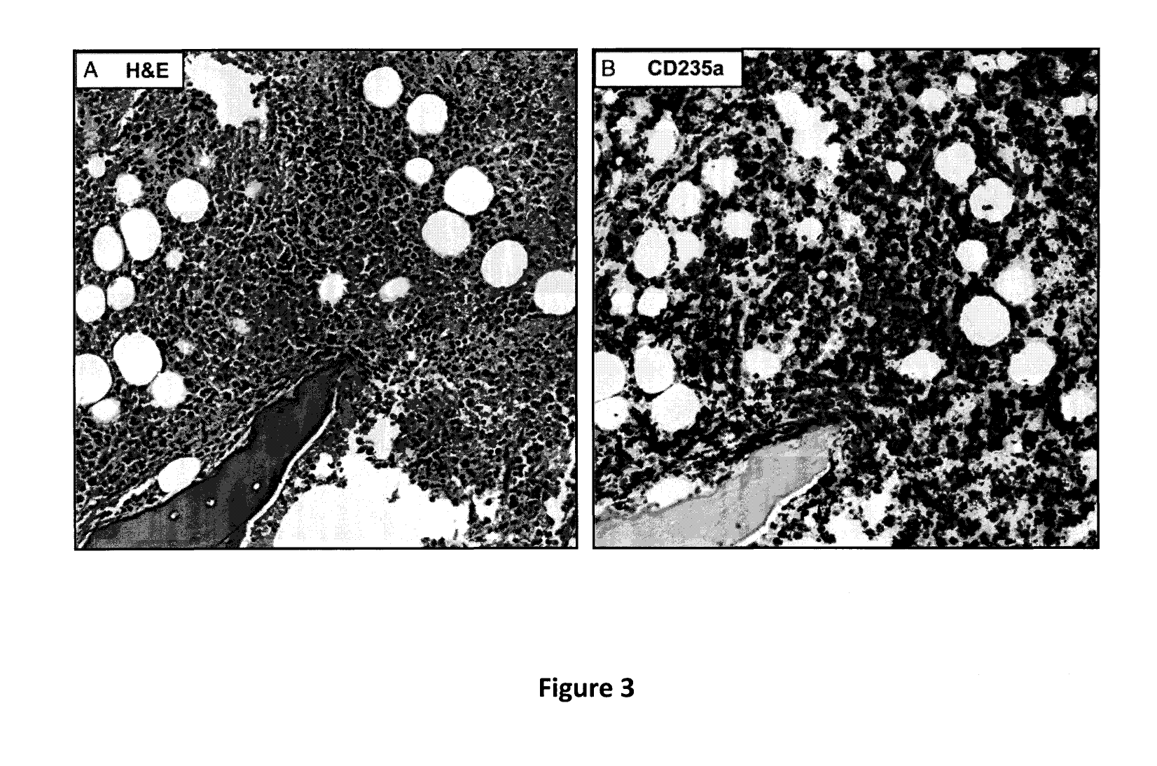

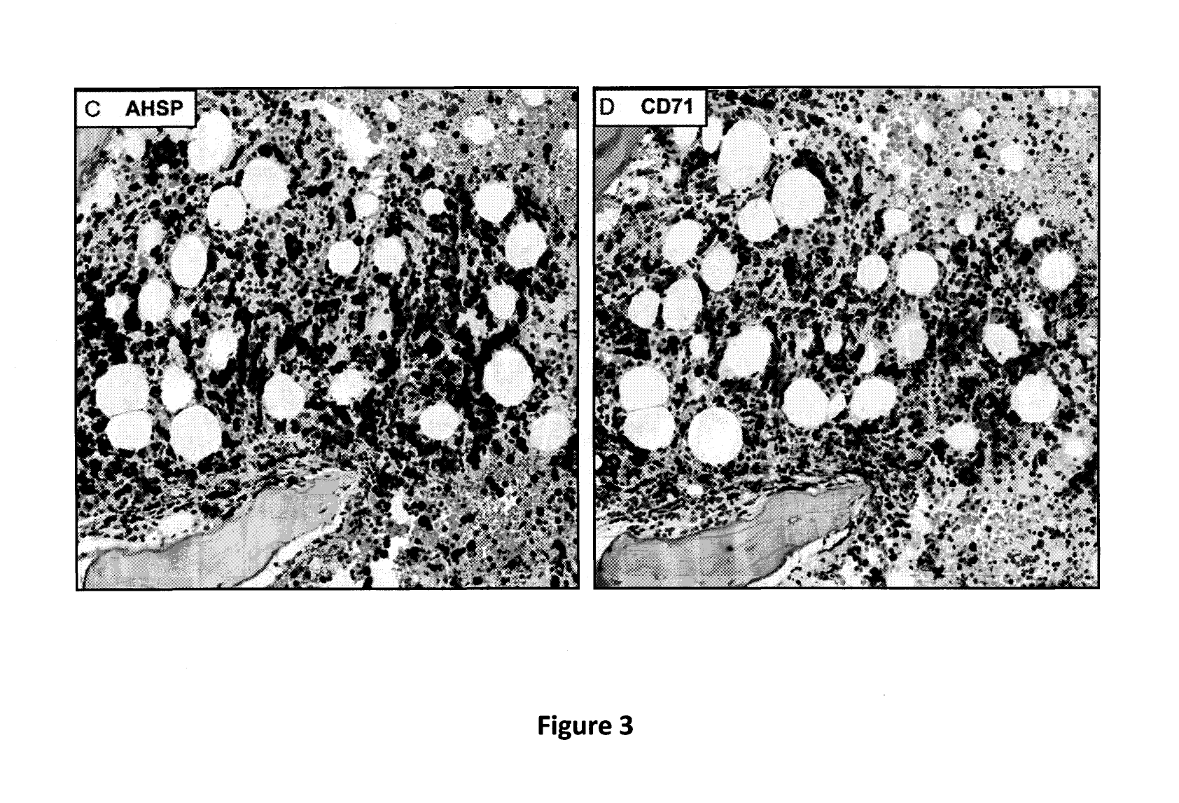

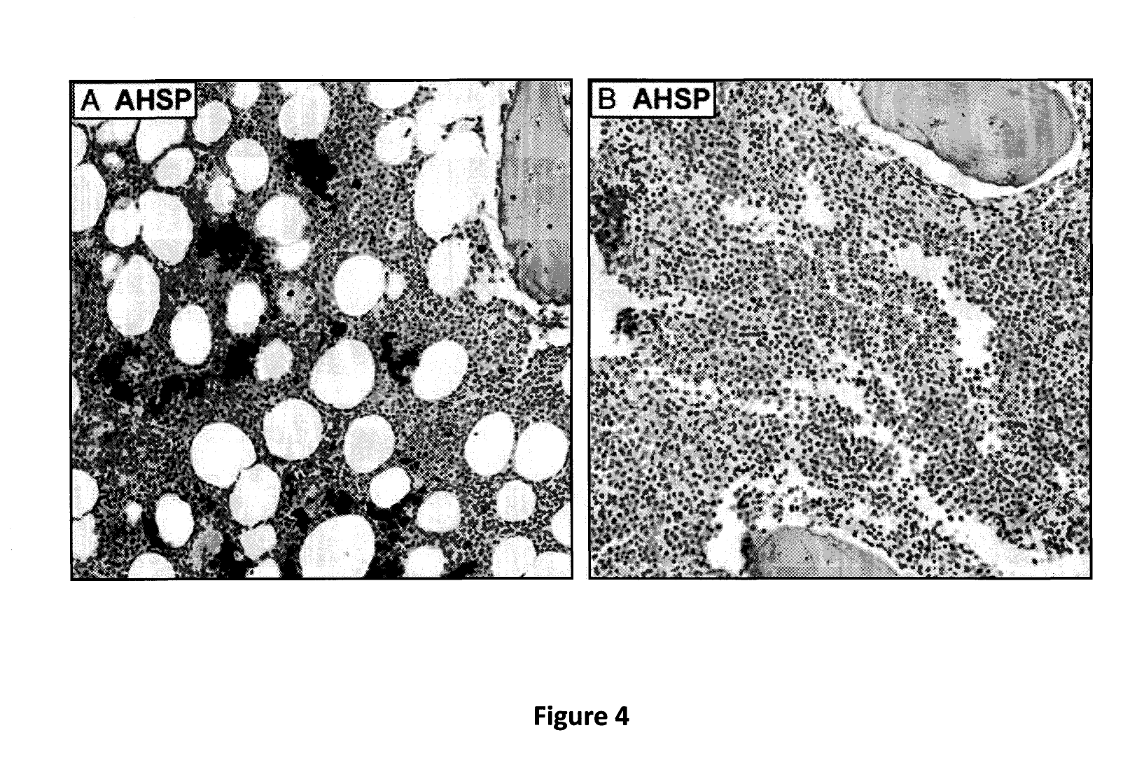

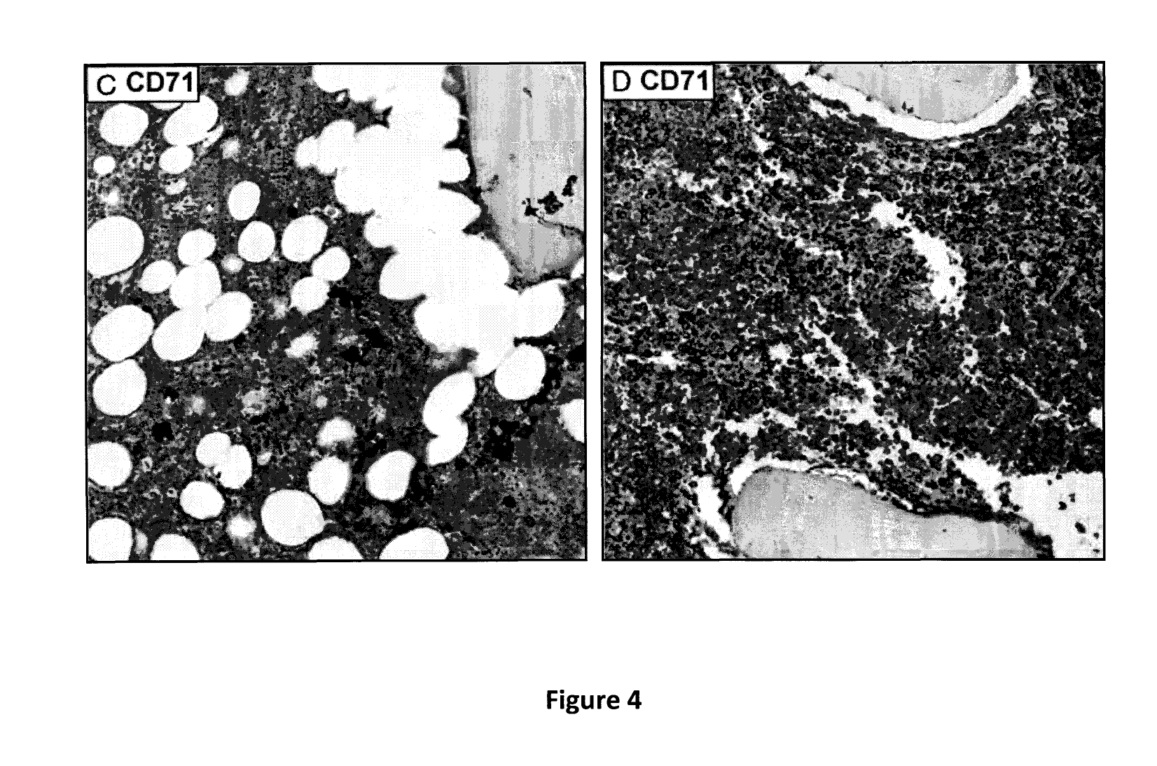

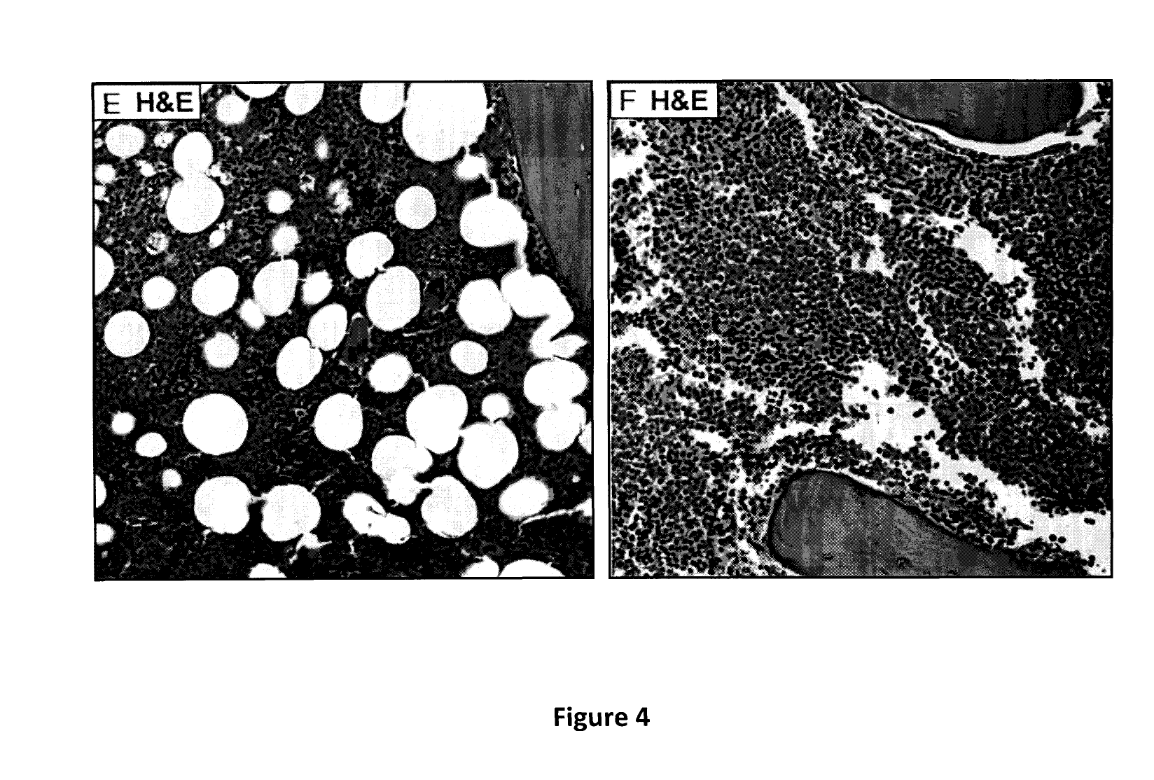

AHSP specifically stained EPs in all normal bone marrow biopsies, all bone marrow biopsies with relative erythroid hyperplasia, and in all adult splenic samples with extramedullary hematopoiesis (FIG. 2). CD71 and CD235a also stained EPs in all of these specimens. In addition, acute erythroid leukemia blasts (both erythroleukemic and pure erythroid leukemic blasts) were positive for AHSP staining (FIG. 3). CD71 and CD235a also stained erythroid blasts in all cases of acute erythroid leukemia. However, CD71 also stained blasts to varying degrees in every morphologic subset of acute myeloid leukemia (FIG. 4) and both acute T-lymphoblastic and B-lymphoblastic leukemia. In general, non-M6 acute myeloid leukemias stained less intensely with CD71 than did EPs. However, a minority of myeloid blasts stained strongly for this antigen at an intensity approximating that exhibited by EPs.

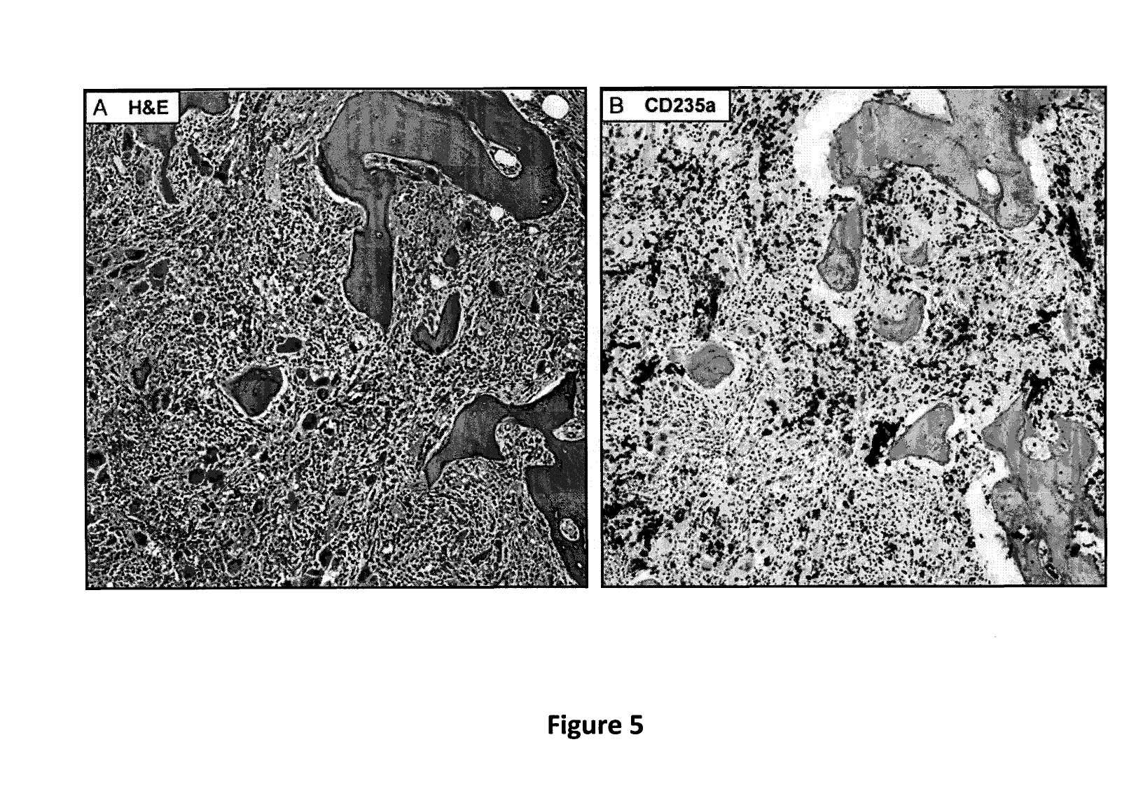

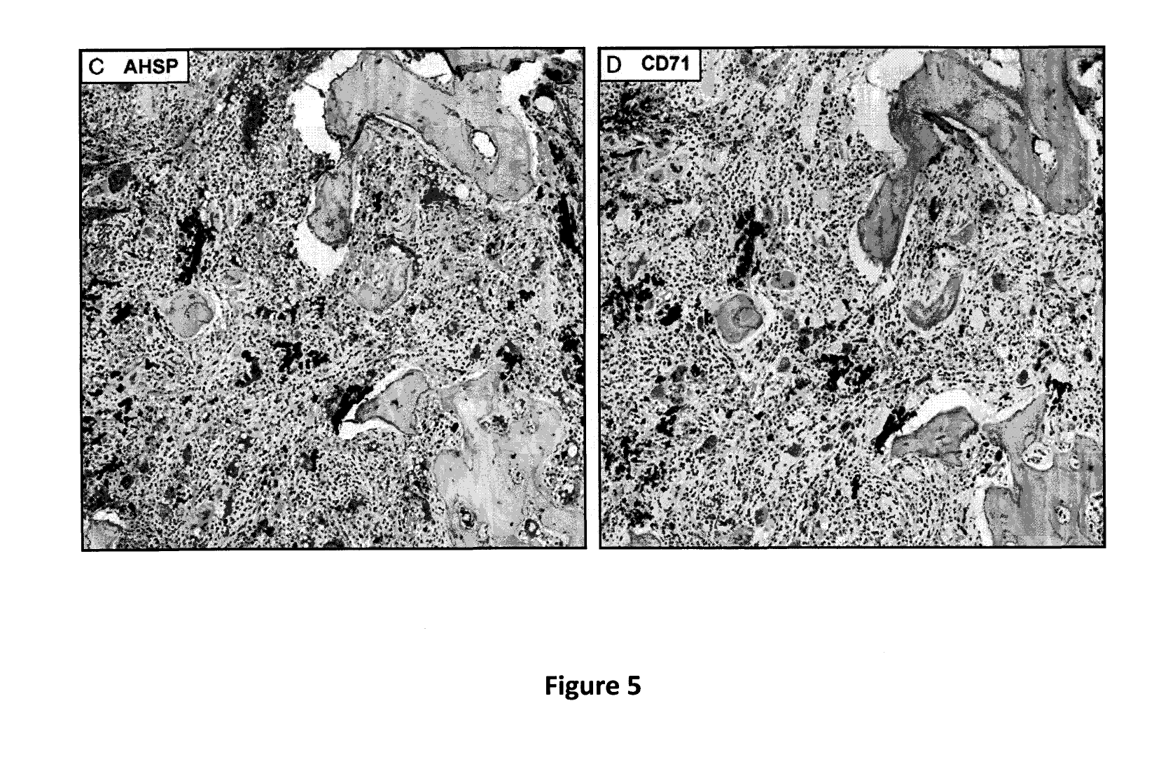

AHSP and CD71 antibodies performed comparably in specimens with myeloproliferative neoplasms and myelodysplastic syndromes, with both antibodies staining EPs in all bone marrow biopsies examined. All cases of primary myelofibrosis exhibited mildly increased staining of megakaryocytes by both AHSP and CD71; CD235a staining did not stain megakaryocytes (FIG. 5). AHSP staining in megakaryocytes was variable between cases and between individual megakaryocytes within the same case.

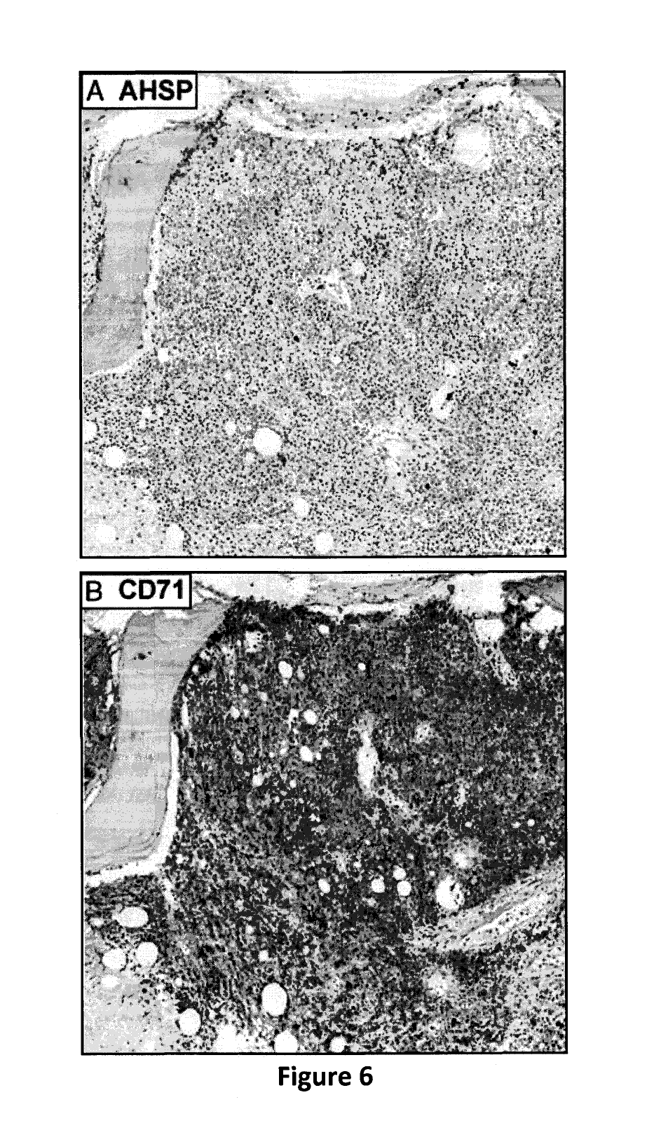

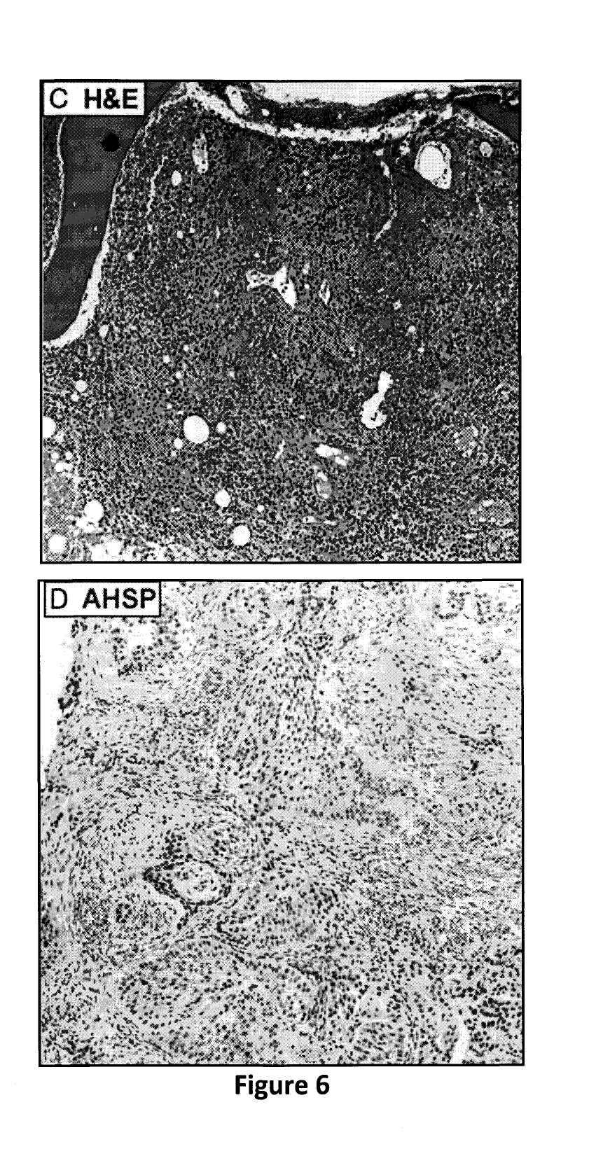

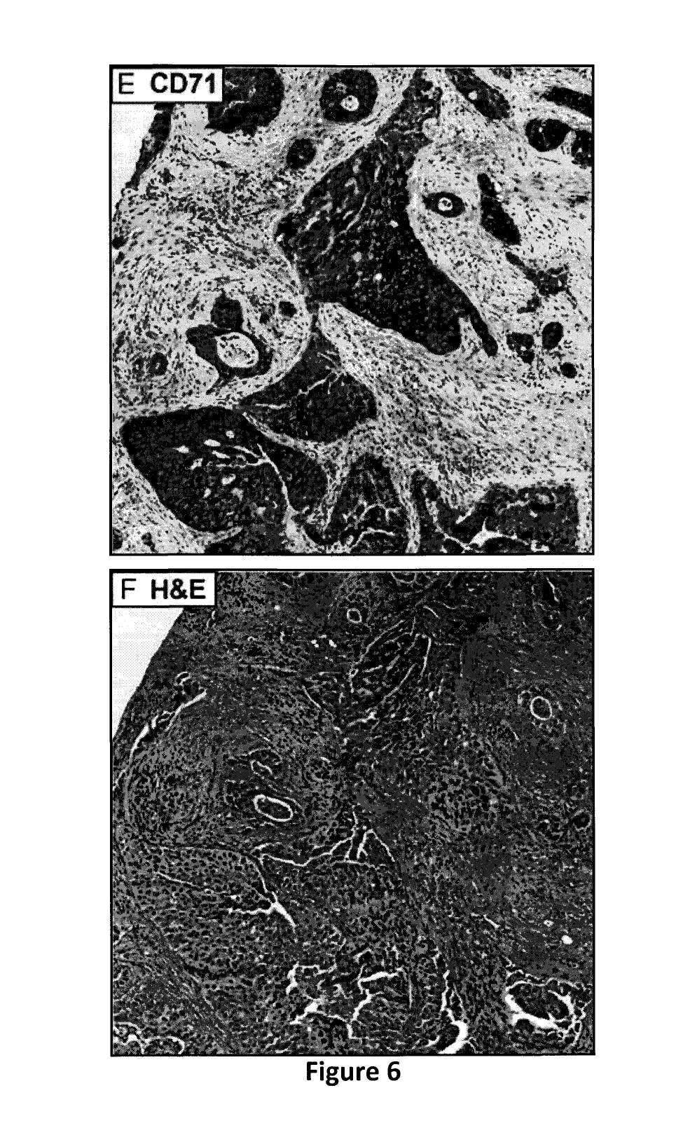

CD71 expression was also noted in many bone marrow biopsies with metastatic malignancies, including DLBCL (4 of 5 cases, FIGS. 6A-C) and carcinoma (2 of 4 cases, FIGS. 6D-F) in adults, and neuroblastoma (5 of 5 cases) and rhabdomyosarcoma (1 of 3 cases) in children. CD71 staining ranged in intensity from dim to bright in metastatic lesions, but was equal to that observed in EPs in multiple cases. CD235a stained both EPs and normucleated RBCs and did not stain metastatic lesions.

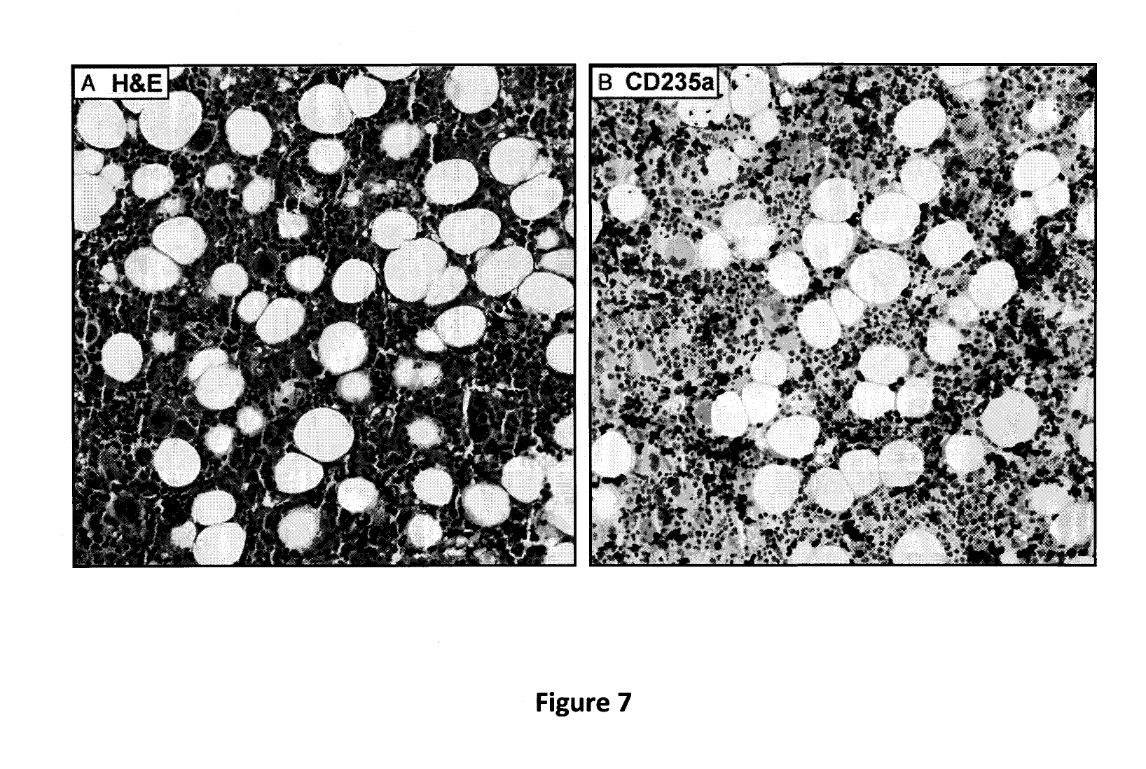

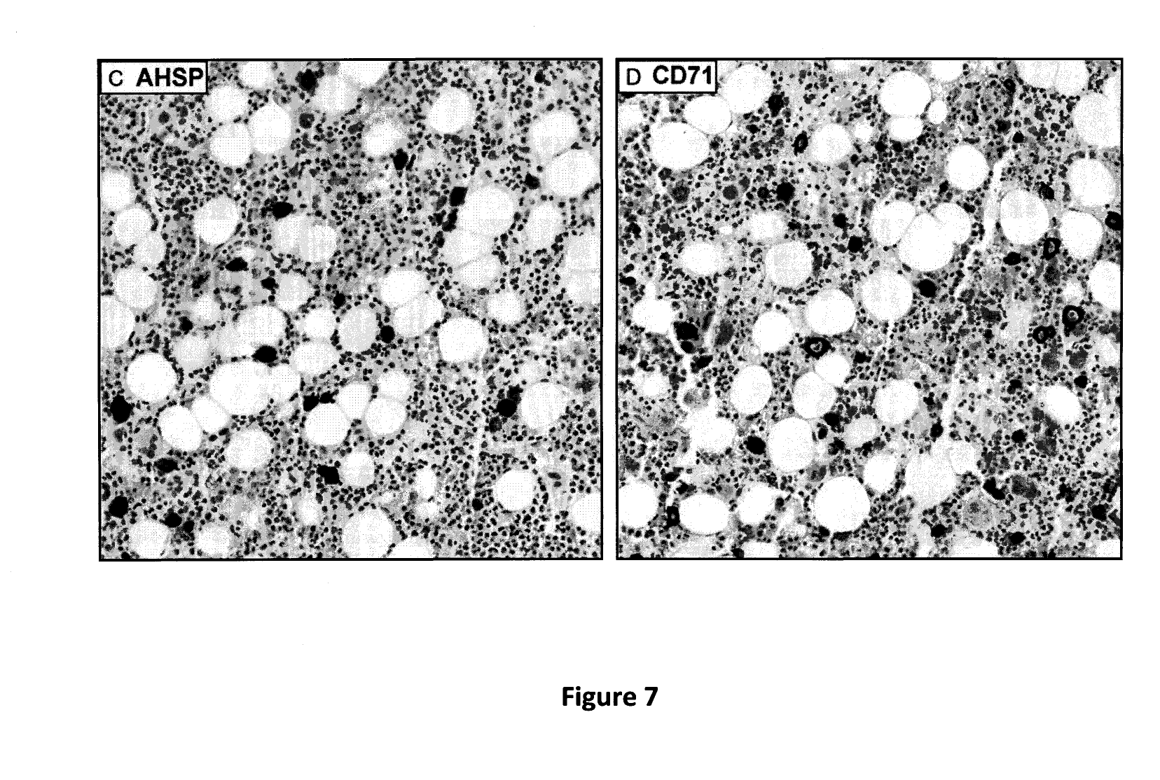

Both AHSP and CD71 stained giant pronormoblasts in bone marrow biopsies from patients with parvovirus infection (FIG. 7), whereas CD235a was negative in these cells, consistent with CD235a expression relatively late in erythroid development.

AHSP and CD71 both stained <1% of normucleated RBCs in all tested specimens, likely marking young reticulocytes that had recently extruded their nucleus. No increase in the frequency of positively staining non-nucleated RBCs was detected in biopsies with myelodysplasia stained with AHSP or CD71. In rare instances, AHSP-stained slides exhibited increased levels of background signal that was localized to noncellular proteinaceous fluid present in the specimens. This artifact was infrequent and was not overrepresented in any individual class of diagnoses. This finding is attributed to nonspecific binding of the polyclonal antibody to serum proteins and did not affect interpretation of cellular staining patterns.

The results presented herein demonstrate that AHSP is a lineage-specific marker of nucleated EPs with improved specificity and equivalent sensitivity as compared with CD71 and CD235a. AHSP staining characteristics did not differ after various fixation protocols (B5, acetic acid-zinc-formalin, and formalin) and were not affected by routine decalcification procedures. In virtually every case, AHSP antibody stained only EPs and erythroid malignancies, whereas CD71 stained malignant cells in a subset of nonerythroid acute leukemias, DLBCL, and metastatic nonhematopoietic malignancies. This result is consistent with the biological restriction of AHSP to the erythroid lineage as a necessary factor for the effective production of Hb.

In contrast, CD71 is a widely expressed protein involved in iron acquisition for most cell types. CD71 expression levels are particularly high in EPs to supply adequate levels of iron for Hb production. However, many rapidly dividing cells, including malignant ones, also exhibit relatively high levels of CD71 in order to supply iron to meet metabolic demands (Ponka et al. (1999) Int. J. Biochem. Cell Biol., 31:1111-1137). Although CD71 and AHSP stain nucleated EPs, analysis of CD235a staining patterns is confounded by staining of anucleate RBCs that frequently contaminate pathologic specimens. This effect is most pronounced in samples of splenic extramedullary hematopoiesis and in bone marrow biopsies with extensive hemorrhage. Thus, among current antigens used for detecting EPs in pathologic samples, only AHSP meets 2 important criteria: high specificity for the erythroid lineage and lack of staining in anucleate RBCs. These properties reflect the biological functions of AHSP. As a molecular chaperone for α-globin, AHSP expression coincides with Hb synthesis, which occurs specifically in erythroid tissues (Kihm et al. (2002) Nature 417:758-763; Weiss et al. (2005) Ann. N.Y. Acad. Sci., 1054:103-117). As Hb synthesis declines during the reticulocyte stage of erythropoiesis, AHSP is no longer needed, and the protein is degraded.

Several other antibodies or antibody panels have been characterized as potential markers of EPs, highlighting the clinical need for such a stain. All previously described markers other than CD235a stain non-EPs in bone marrow biopsies. A panel of 3 immunohistochemical markers targeting proteins whose expression is not limited to the erythroid lineage has also recently been described (Rollins-Raval et al. (2012) Am. J. Clin. Pathol., 137:30-38). Although this panel of markers sensitively stains for EPs, it also marked blasts in a subset of nonerythroid acute leukemias. E-cadherin has also been shown to be expressed in EPs, although its expression is present in many epithelial cell types and also is not expressed in erythroleukemia (Acs et al. (2001) Arch. Pathol. Lab Med., 125:198-201; Armeanu et al. (1995) J. Cell Biol., 131:243-249). CD36, the thrombospondin receptor, is expressed not only in EPs but also in other cell types, including platelets, macrophages, and endothelial cells (Febbraio et al. (2001) J. Clin. Invest., 108:785-791; Filippone et al. (2010) PLoS One 5:e9496.).

CD71 has been previously reported to label blasts in a subset of non-erythroid acute leukemias in some studies but not in others (Dong et al. (2011) Am. J. Surg. Pathol., 35:723-732; Marsee et al. (2010) Am. J. Clin. Pathol., 134:429-435). The results presented herein confirm these findings. Although CD71 expression is variable in nonerythroid malignancies, certain cases demonstrated CD71 expression in blasts that approximated levels exhibited by EPs. AHSP did not stain leukemic blasts in any cases of nonerythroid acute leukemias. AHSP staining was also limited to EPs in all cases of marrow involvement by DLBCL or nonhematopoietic malignancies, whereas CD71 stained the neoplastic cells in many of these cases. CD71 expression has been reported in a wide variety of activated or proliferating cell types, concordant with its biological role as the transferrin receptor. CD71 expression is likely upregulated in these cases secondary to increased iron demand in these rapidly proliferating cells.

Detection of AHSP in megakaryocytes associated with primary myelofibrosis is of uncertain etiology. Non-specific staining is unlikely, as the antibody does not stain normal megakaryocytes in control specimens or bone marrow affected by B-cell lymphoma or parvovirus. It is possible that the pathologic megakaryocytes in primary myelofibrosis aberrantly express AHSP and occasionally CD71 as well, reflecting dysmegakaryopoiesis with derepression of an erythroid gene expression program. Erythroid and megakaryocytic lineages derive from a common bipotential progenitor and express overlapping sets of hematopoietic transcription factors (Pang et al. (2005) J. Clin. Invest., 115:3332-3338). Megakaryocytes in primary myelofibrosis have been demonstrated to aberrantly express multiple genes and miRNAs; however, AHSP has never been specifically studied (Hussein et al. (2009) Platelets 20:391-400; Theophile et al. (2008) Exp. Hematol., 36:1728-1738). Importantly, AHSP did not stain blasts in acute megakaryocytic leukemia, whereas CD71 marked them brightly.

AHSP and CD71 both demonstrated intense staining of giant pronormoblasts in cases of parvovirus infection, whereas CD235a does not. Negative CD235a expression and CD71 positivity in infected giant pronormoblasts has been reported (Sadahira et al. (2001) Int. J. Hematol., 74:147-152; Dong et al. (2011) Am. J. Surg. Pathol., 35:723-732). AHSP positivity in giant pronormoblasts confirms the ability of AHSP to mark EPs that are in the earliest stages of the erythroid lineage.

In addition to its clinical utility as a specific immunohistochemical marker of EPs, AHSP expression may also be used in other clinical and research settings. For example, AHSP may be used as an intracellular flow cytometric marker to identify acute erythroid leukemias and may be used to help differentiate between morphologic subtypes (erythroleukemia vs. acute erythroid leukemia).

In summary, AHSP is a novel marker of EPs whose biological role as a chaperone protein necessary for Hb formation confers lineage specificity and whose expression is limited to nucleated EPs. AHSP will be useful in many clinical scenarios including assigning lineage to neoplastic or reactive immature cells and identifying EPs in dyspoietic marrows.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method of determining the presence of a blood disorder in a subject, said method comprising:
a) contacting a biological sample obtained from said subject with at least one antibody or antibody fragment immunologically specific for alpha-hemoglobin stabilizing protein (AHSP); and
b) detecting cells bound by said AHSP antibody, wherein an alteration in at least one morphological feature of said biological sample from the subject compared to a biological sample obtained from a normal subject is indicative of said blood disorder.

2. The method of claim 1, wherein said AHSP antibody is conjugated to at least one detection agent.

3. The method of claim 2, wherein said detection agent is selected from the group consisting of isotopes, radioisotopes, imaging agents, fluorescent agents, and contrast agents.

4. The method of claim 1, wherein said blood disorder is selected from the group consisting of anemias, leukemias, and myelodysplasias.

5. The method of claim 4, wherein said blood disorder is a myelodyplastic syndrome.

6. The method of claim 1, wherein said morphological feature is selected from the group of the relative abundance of nucleated erythroid precursors, the spatial arrangement of nucleated erythroid precursors, cellularity, and size, morphology, or cellular architecture of erythroid cluster.

7. The method of claim 1, wherein said biological sample is bone marrow.

8. The method of claim 1, wherein said biological sample is placental and wherein increased placental nucleated erythroid precursors are indicative of perinatal hypoxia.

9. The method of claim 1, further comprising contacting the biological sample with at least one other diagnostic antibody.

10. A method of detecting erythroid precursor cells comprising:
a) contacting a population of cells with at least one antibody or antibody fragment immunologically specific for alpha-hemoglobin stabilizing protein (AHSP); and
b) detecting cells bound by said AHSP antibody, wherein said cells bound by the AHSP antibody are erythroid precursor cells.

11. The method of claim 10, wherein said AHSP antibody is conjugated to at least one detection agent.

12. The method of claim 11, wherein said detection agent is selected from the group consisting of isotopes, radioisotopes, imaging agents, fluorescent agents, and contrast agents.

13. The method of claim 10, wherein said population of cells are in a biological sample obtained from a subject.

14. The method of claim 10, further comprising contacting the population of cells with at least one other diagnostic antibody.

15. A kit comprising at least one antibody or antibody fragment immunologically specific for alpha-hemoglobin stabilizing protein (AHSP) and at least one other diagnostic antibody.

16. The kit of claim 15, wherein said diagnostic antibody is an antibody immunologically specific for CD235, CD71, hemoglobin, or myeloperoxidase (MPO).

17. The kit of claim 15, wherein said kit further comprises hemalum and eosin Y.


SEQUENCE LISTING (1 sequence)

SEQ ID NO: 1 - protein, 102 aa, Homo sapiens
MALLKANKDLISAGLKEFSVLLNQQVFNDPLVSEEDMVTVVEDWMNFYINYYRQQVTGEPQERDKALQELRQELNTLANPFLAKYRDFLKSHELPSHPPPSS